(19) United States
(12) Patent Application Publication
(10) Pub. No.: US 2020/0129250 A1
(43) Pub. Date: Apr. 30, 2020

(54) ROBOTIC SURGICAL CART

(71) Applicant: SS Innovations China Co., Ltd., Hangzhou (CN)

(72) Inventors: Salman KAPADIA, Barapatthar (IN); Manish, Chandigarh (IN)

(73) Assignee: SS Innovations China Co., Ltd., Hangzhou (CN)

(21) Appl. No.: 16/659,067

(22) Filed: Oct. 21, 2019

(30) Foreign Application Priority Data
Oct. 31, 2018 (IN) 201811041109

(51) Int. Cl. A61B 34/30; A61B 50/13; A61B 34/00; A61B 90/50; B25J 13/06; B25J 18/02

(52) U.S. Cl. CPC A61B 34/30; A61B 50/13; A61B 34/25; A61B 2017/00991; B25J 13/06; B25J 18/025; A61B 90/50

(57) ABSTRACT

A robotic surgical cart (300) for a robotic arm (201) is disclosed herein. The robotic surgical cart (300) comprises a column (401) having a first end (403) and a second end (405). The robotic surgical cart (300) further comprises a top plate (407) affixed on the first end (403) of the column (401), where the top plate (407) is configured to mount the robotic arm (201) thereon, and at least one guiding shaft (411) affixed on one end to the top plate (407), that provides a controlled channel for movement of the robotic arm (201).The robotic surgical cart (300) further comprises a telescopic pillar (415) including two or more tubular members (417, 419), affixed below the top plate (407). The telescopic pillar (415) defines a longitudinal axis to provide upward and downward movement of the two or more tubular members (417, 419) in the channel provided by the at least one guiding shaft (411) such that the telescopic pillar (415) moves the robotic arm (201) in a position in which a portion of the robotic arm (201) is exposed to contact a patient on a surgical table.

### TECHNICAL FIELD

The present disclosure generally relates to a robotic surgical system for minimally invasive surgery. More particularly, the disclosure relates to an improved robotic surgical cart in the robotic surgical system.

### BACKGROUND

This section is intended to introduce the reader to various aspects of art that may be related to various aspects of the present disclosure, which are described below. This disclosure is believed to be helpful in providing the reader with background information to facilitate a better understanding of the various aspects of the present disclosure. Accordingly, it should be understood that these statements are to be read in this light, and not just as admissions of prior art.

Robotically assisted surgical systems have been adopted worldwide to replace conventional surgical procedures to reduce amount of extraneous tissue(s) that may be damaged during surgical or diagnostic procedures, thereby reducing patient recovery time, patient discomfort, prolonged hospital tenure, and particularly deleterious side effects. In robotically assisted surgeries, the surgeon typically operates a master controller at a surgeon console to seamlessly capture and transfer complex actions performed by the surgeon giving the perception that the surgeon is directly articulating surgical tools to perform the surgery. The surgeon operating on the surgeon console may be located at a distance from a surgical site or may be located within an operating theatre where the patient is being operated.

The robotically assisted surgeries have revolutionized the medical field and one of the fastest growing sectors in medical device industry. However, the major challenge in robotically assisted surgeries is to ensure the safety and precision during the surgery. One of the key areas of robotically assisted surgeries is the development of surgical robots for minimally invasive surgery. Over the last couple of decades, surgical robots have evolved exponentially and has been a major area of innovation in the medical device industry.

The robotically assisted surgical systems comprises multiple robotic arms aiding in conducting robotic surgeries. The robotically assisted surgical system utilizes a sterile barrier to separate the non-sterile section of the robotic arm from a mandatory sterile surgical instrument attached to the robotic arm at an operating end. The sterile barrier may include a sterile plastic drape that envelops the robotic arm and a sterile adapter that operably engages with the sterile surgical instrument in a sterile field.

Traditionally, the robotically assisted surgical systems comprises one or more robotic arms that are loaded together on one cart. These carts are generally heavy to maneuver and requires a large area to install. Also, when a robotic arm becomes damaged or require maintenance, it is often difficult and costly to repair and may also cause significant delays in surgical operations.

Further, these carts are heavy and can pick up a substantial amount of momentum during transportation such that it may not be easy for a user to steer the cart to avoid objects and/or to slow down the cart when approaching the operating table. In such instances, if the robotic arm contacts the operating table or some other object at a high velocity, the robotic arm and/or the operating table may become damaged due shock or impact forces resulting from the contact.

Another challenge in the robotically assisted surgical system is positioning of the robotics arms from the patient table and managing an optimum height of the robotic arms to perform the surgical operations successfully. In currently used robotics surgical carts, since all the arms are attached to a single cart or column, positioning and managing the proper height from the patient bed and calculating the distance from each arm to avoid collision of arms is difficult to maintain and may causes manual intervention to correct the difference which is generally calculated by a software which also increase manual labor and delays the surgical operation.

In the light of aforementioned challenges, there is a need for a modular robotic surgical cart with improved structure such that all the issues relating with the current robotic surgical carts are resolved.

### SUMMARY

The present disclosure seeks to provide an improved robotic surgical cart in the robotic surgical system.

In one aspect, an embodiment of the present disclosure provides a robotic surgical cart for a robotic arm. The robotic surgical cart comprises a column having a first end and a second end. The robotic surgical cart further comprises a top plate) affixed on the first end of the column, where the top plate is configured to mount the robotic arm thereon, and at least one guiding shaft affixed on one end to the top plate, that provides a controlled channel for movement of the robotic arm. The robotic surgical cart (300) further comprises a telescopic pillar including two or more tubular members, affixed below the top plate. The telescopic pillar defines a longitudinal axis to provide upward and downward movement of the two or more tubular members in the channel provided by the at least one guiding shaft such that the telescopic pillar moves the robotic arm in a position in which a portion of the robotic arm is exposed to contact a patient on a surgical table.

Optionally, the two or more tubular members coaxial with one another and telescopically engaged one inside the other.

Optionally, the two or more tubular members are configured to move upward and downward within a range of 1 mm to 150 mm.

Optionally, the robotic surgical cart further comprises a user interface pivotally affixed to the first end of the column, wherein the user interface provides the controls for the movement of the robotic arm and wherein the user interface comprises a screen displaying the height of the robotic surgical cart and the distance from the surgical table.

Optionally, the column comprises at least one shaft and at least one stabilizing plate joined together with the shaft at an end.

Optionally, the robotic surgical cart further comprises a control box affixed within the column, wherein the control box is configured to provide power to the telescopic pillar.

Optionally, the robotic surgical cart further comprises a secondary battery affixed to the second end of the column, wherein the secondary battery is a backup battery to power the telescopic pillar in case of a failure of the control box.

Optionally, the robotic surgical cart further comprises an outer casing enclosing the column and the other components of the robotic surgical cart.

Optionally, the robotic surgical cart further comprises a handle bar pivotally affixed to the first end of the column, wherein the handle has a U-shaped structure and configured to allow a user to move the robotic surgical cart from one position to another position.

Optionally, the robotic surgical cart further comprises a footprint ring affixed on the second end of the column, wherein the footprint ring provides a platform to the robotic surgical cart.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of the disclosure, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1(a) illustrates a schematic diagram of multiple robotic arms of a robotic surgical system in accordance with an embodiment of the disclosure;

FIG. 1(b) illustrates a schematic diagram of a surgeon console of the robotic surgical system in accordance with an embodiment of the disclosure;

FIG. 1(c) illustrates a schematic diagram of a vision cart of the robotic surgical system in accordance with an embodiment of the disclosure;

FIG. 2 illustrates a perspective view of a tool interface assembly mounted on a robotic arm in accordance with an embodiment of the disclosure;

FIG. 3(a) illustrates a perspective view of a robotic surgical cart in accordance with an embodiment of the disclosure;

FIG. 3(b) illustrates a perspective view of the robotic surgical cart having a mounted robotic arm in accordance with an embodiment of the disclosure;

FIG. 3(c) illustrates a perspective view of the robotic surgical cart without an outer casing in accordance with an embodiment of the disclosure;

FIG. 4(a) illustrates an exploded view of the robotic surgical cart in accordance with an embodiment of the disclosure;

FIG. 4(b) illustrates a cut-out view of the robotic surgical cart in accordance with an embodiment of the disclosure;

FIG. 4(c) illustrates a top view of an omni-directional wheels along with an actuator in accordance with an embodiment of the disclosure;

FIG. 4(d) illustrates a bottom view of the omni-directional wheels in accordance with an embodiment of the disclosure;

FIG. 5(a) illustrates a frontal view of a column of the robotic surgical cart in accordance with an embodiment of the disclosure; and

FIG. 5(b) illustrates a perspective view of the robotic surgical cart along with the various components in accordance with an embodiment of the disclosure.

### DETAILED DESCRIPTION OF DISCLOSURE

For the purpose of promoting an understanding of the principles of the disclosure, reference will now be made to the embodiment illustrated in the drawings and specific language will be used to describe the same. It will nevertheless be understood that no limitation of the scope of the disclosure is thereby intended, such alterations and further modifications in the illustrated system, and such further applications of the principles of the disclosure as illustrated therein being contemplated as would normally occur to one skilled in the art to which the disclosure relates.

It will be understood by those skilled in the art that the foregoing general description and the following detailed description are exemplary and explanatory of the disclosure and are not intended to be restrictive thereof. Throughout the patent specification, a convention employed is that in the appended drawings, like numerals denote like components.

Reference throughout this specification to “an embodiment”, “another embodiment”, “an implementation”, “another implementation” or similar language means that a particular feature, structure, or characteristic described in connection with the embodiment is included in at least one embodiment of the present disclosure. Thus, appearances of the phrase “in an embodiment”, “in another embodiment”, “in one implementation”, “in another implementation”, and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises . . . a” does not, without more constraints, preclude the existence of other devices or other sub-systems or other elements or other structures or additional devices or additional sub-systems or additional elements or additional structures.

Unless otherwise defined, all technical and scientific terms used herein have the same meaning as commonly understood by one of ordinary skill in the art to which this disclosure belongs. The apparatus, system, and examples provided herein are illustrative only and not intended to be limiting.

The terms “a” and “an” herein do not denote a limitation of quantity, but rather denote the presence of at least one of the referenced item. Further, the term sterile barrier and sterile adapter denotes the same meaning and may be used interchangeably throughout the description.

Embodiments of the disclosure will be described below in detail with reference to the accompanying drawings.

The disclosure relates to a robotic surgical system for minimally invasive surgery. The robotic surgical system will generally involve the use of multiple robotic arms. One or more of the robotic arms will often support a surgical tool which may be articulated (such as jaws, scissors, graspers, needle holders, micro dissectors, staple appliers, tackers, suction/irrigation tools, clip appliers, or the like) or non-articulated (such as cutting blades, cautery probes, irrigators, catheters, suction orifices, or the like). One or more of the robotic arms will often be used to support one or more surgical image capture devices such as an endoscope (which may be any of the variety of structures such as a laparoscope, an arthroscope, a hysteroscope, or the like), or optionally, some other imaging modality (such as ultrasound, fluoroscopy, magnetic resonance imaging, or the like).

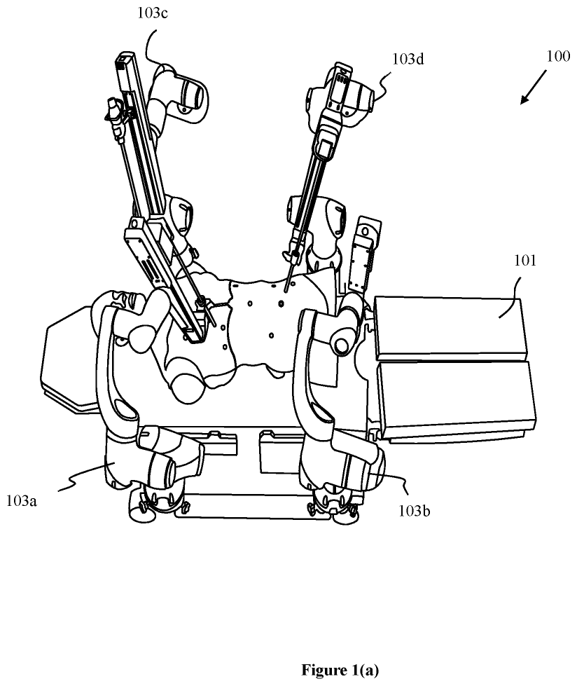

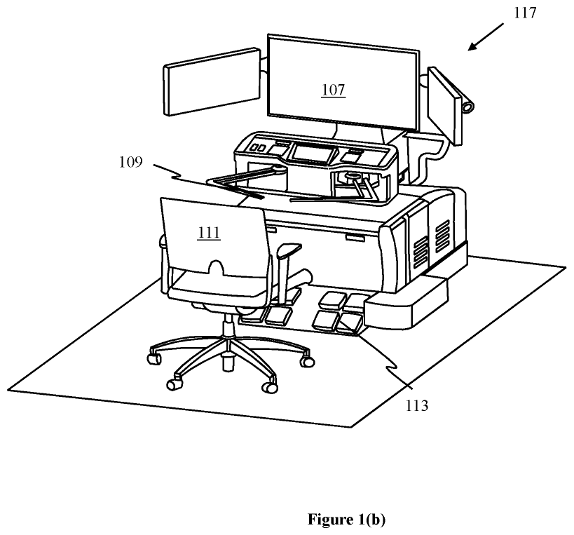

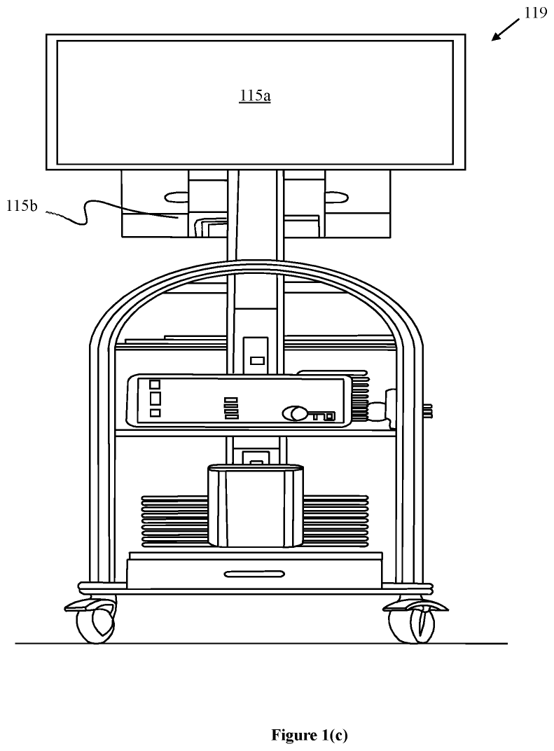

FIG. 1(a) illustrates a schematic diagram of multiple robotic arms of a robotic surgical system in accordance with an embodiment of the disclosure. Specifically, FIG. 1 illustrates the robotic surgical system (100) having four robotic arms (103a), (103b), (103c), (103d) mounted around a patient cart (101). The four-robotic arms (103a), (103b), (103c), (103d) as depicted in FIG. 1 is for illustration purpose and the number of robotics arms may vary depending upon the type of surgery or the robotic surgical system. The four robotic arms (103a), (103b), (103c), (103d) are mounted along the patient cart (101) and may be arranged in different manner but not limited to the robotic arms (103a), (103b), (103c), (103d) mounted on the patient cart 101 or the robotic arms (103a), (103b), (103c), (103d) separately mounted on a movable means or the robotic arms (103a), (103b), (103c), (103d) mechanically and/or operationally connected with each other or the robotic arms (103a), (103b), (103c), (103d) connected to a central body such that the robotic arms (103a), (103b), (103c), (103d) branch out of the central body (now shown).

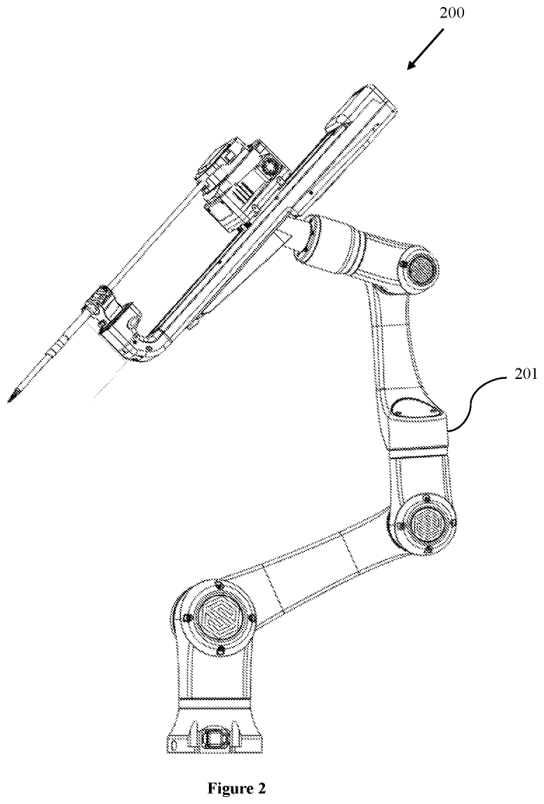

FIG. 1(b) illustrates a schematic diagram of a surgeon console of the robotic surgical system in accordance with an embodiment of the disclosure. The surgeon console (117) aids the surgeon to remotely operate the patient lying on the patient cart (101) by controlling the robotic arms (103a), (103b), (103c), (103d) inside the body of the patient. The surgeon console (117) is configured to control the movement of surgical instruments (as shown in FIG. 2) while the instruments are inside the patient. The surgeon console (117) may comprise of at least an adjustable viewing means (107) but not limited to 2D/3D monitors, wearable viewing means (not shown) and in combination thereof. The surgeon console (117) may be equipped with multiple displays which would not only show 3D high definition (HD) endoscopic view of a surgical site at the patient cart (101) but may also shows additional information from various medical equipment's which surgeon may use during the robotic surgery. Further, the viewing means (107) may provide various modes of the robotic surgical system (100) but not limited to identification and number of robotic arms attached, current tool type attached, current tool tip position, collision information along with medical data like ECG, ultrasound display, fluoroscopic images, CT, MRI information. The surgeon console (117) may further comprise of mechanism for controlling the robotics arms but not limited to one or more hand controllers (109), one or more foot controllers (113), a clutch mechanism (not shown), and in combination thereof. The hand controllers (109) at the surgeon console (117) are required to seamlessly capture and transfer complex actions performed by surgeon giving the perception that the surgeon is directly articulating the surgical tools. The different controllers may require for different purpose during the surgery. In some embodiments, the hand controllers (109) may be one or more manually-operated input devices, such as a joystick, exoskeletal glove, a powered and gravity-compensated manipulator, or the like. These hand controllers (109) control teleoperated motors which, in turn, control the movement of the surgical instruments attached to the robotic arms. The surgeon may sit on a resting apparatus such as a chair (111), as depicted in FIG. 1(b), while controlling the surgeon console (117). The chair (111) may be adjustable with means in height, elbow rest and the like according to the ease of the surgeon and also various control means may be provided on the chair (111). Further, the surgeon console (117) may be at a single location inside an operation theatre, or may be distributed at any other location in the hospital provided connectivity to the robotics arms is maintained.

FIG. 1(c) illustrates a schematic diagram of a vision cart of the robotic surgical system in accordance with an embodiment of the disclosure. The vision cart (119) is configured to display the 2D and/or 3D view of the operation captured by an endoscope. The vision cart (119) may be adjusted at various angles and heights depending upon the ease of view. The vison cart (119) may have various functionality but not limited to providing touch screen display, preview/recording/playback provisions, various inputs/outputs means, 2D to 3D converters and the like. The vision cart (119) may include a vision system portion (not shown) that enables a spectator or other non-operating surgeons to view a surgical site from outside the patient's body. One of the robotics arms typically engage a surgical instrument that has a video-image-capture function (i.e., a camera instrument) for displaying the captured images on the vision cart (119). In some robotic surgical system configurations, the camera instrument includes optics that transfer the images from the distal end of the camera instrument to one or more imaging sensors (e.g., CCD or CMOS sensors) outside of the patient's body. Alternatively, the imaging sensor(s) may be positioned at the distal end of the camera instrument, and the signals produced by the sensor(s) may be transmitted along a lead or wirelessly for processing and display on the vision cart (119).

FIG. 2 illustrates a perspective view of a tool interface assembly mounted on a robotic arm in accordance with an embodiment of the disclosure. The tool interface assembly (200) is mounted on the robotic arm (201) of the robotic surgical system (100). The tool interface assembly (200) is the main component for performing the robotic surgery on a patient. The robotic arm (201) as shown in FIG. 2 is shown for the illustration purpose only and other robotic arms with different configurations, degree of freedom (DOF) and shapes may be used.

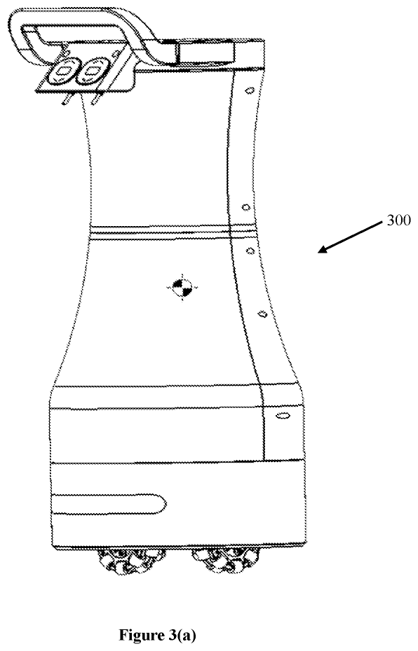

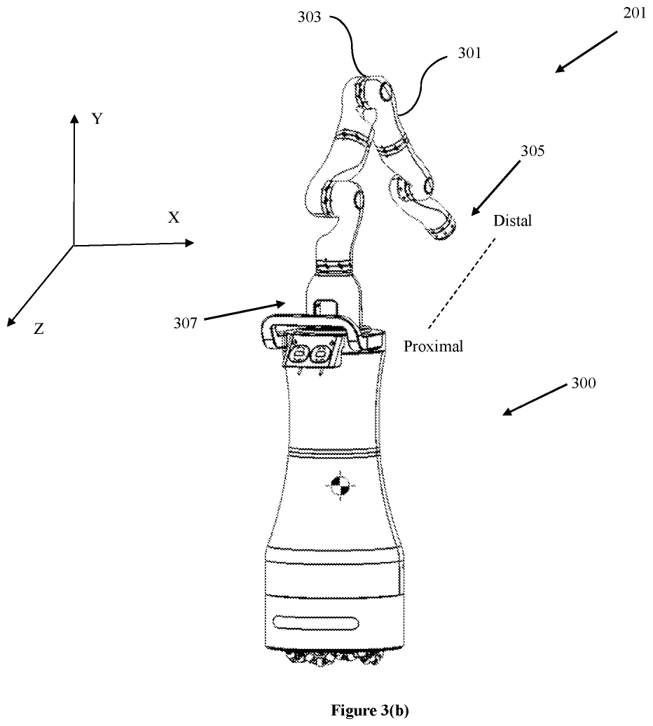

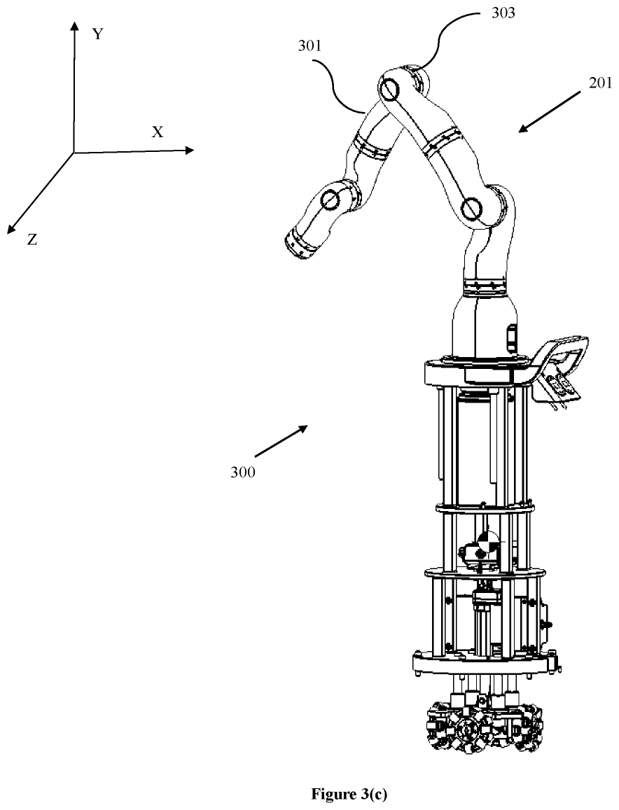

FIG. 3(a) illustrates a perspective view of a robotic surgical cart in accordance with an embodiment of the disclosure and FIG. 3(b) illustrates a perspective view of a robotic surgical cart having a mounted robotic arm in accordance with an embodiment of the disclosure. The robotic surgical cart (300) is adapted for transporting, delivering, and securing robotic arms to a surgical table having a top on which a patient can be disposed. The robotic surgical cart (300) also protects the attached robotic arm (201) from being damaged during transport and attachment of the robotic arm (201) to the surgical table. The robotic surgical cart (300) may include, for example, a damping apparatus that reduces an impact force imparted to the robotic arm (201) (e.g., absorbs shock imparted to the robotic arm) as a result of the robotic arm (201) coming into contact with the surgical table. Also, the robotic surgical cart (300) is configured to mount the robotic arm (201) on one of its surfaces.

In an embodiment, the robotic surgical cart (300) is positioned proximate to the surgical table such that the robotic arm (201) can easily access the patient on the surgical table. The robotic surgical cart (300) may provide for movement of the robotic arms in at least one of a lateral, longitudinal, or vertical direction relative to the surgical table prior to the placement of the robotic arm (201) to the surgical table.

In a robotically-assisted surgical procedure, one or more robotic arms (201) can be disposed in a desired operative position relative to a patient disposed on the surgical table. The robotic arm(s) can be used to perform a surgical procedure on a patient disposed on the surgical table. In particular, a distal end of each robotic arm (201) can be disposed in a desired operative position so that the tool interface assembly (200) (shown in FIG. 2) coupled to the distal end of the robotic arm (201) can perform a desired operative function.

As shown schematically in FIG. 3(b), the robotic arm (201) may include a distal end (305) and a proximal end (307). The distal end (305) may include or have coupled thereto a medical instrument or tool interface assembly (200) (as shown in FIG. 2). The proximal end (307) may include coupling portions to allow the robotic arm (201) to be coupled to the robotic surgical cart (300). The robotic arm (201) may include two or more link members or segments (301) coupled together at joints (303) that may provide for translation along and/or rotation about one or more of the X, Y and/or Z axes (shown in FIG. 3(b)).

The robotic surgical cart (300) may support the robotic arm (201) in a variety of configurations. In some embodiments, the robotic surgical cart (300) may support the robotic arm (201) such that the center of gravity of the robotic arm (201) is below one or more support structure locations (e.g., cradles) of the robotic surgical cart (300) such that the stability of the robotic arm (201) and the robotic surgical cart (300) is increased. In some embodiments, the robotic surgical cart (300) may support the robotic arm (201) such that the robotic surgical cart (300) bears most or all of the weight of the robotic arm (201) and a coupling mechanism (not shown) of the robotic arm (201) may be manually manipulated by a user without the user bearing the most or all of the weight of the robotic arm (201). For example, the robotic arm (201) may be suspended from a structure of the robotic surgical cart (300) or rested on a base of the robotic surgical cart (300).

Additionally, the robotic surgical cart (300) may include any suitable means for adjusting the height of the robotic surgical cart (300) and/or the robotic arm (201) such that the height of the robotic arms (201) may be adjusted relative to the surgical table. Thus, the robotic surgical cart (300) may move the robotic arm (201) along the X, Y, and/or Z axes and/or rotationally about the X, Y, and/or Z axes such that a coupling portion of the robotic arm (201) may be aligned for engagement with a mating coupling portion on the surgical table.

FIG. 3(c) illustrates a perspective view of the robotic surgical cart without an outer casing in accordance with an embodiment of the disclosure. The above-mentioned figure illustrates the inside view of the robotic surgical cart (300) and its components. Details explanation of the components shall be explained in the accompanying figures.

The robotic arm (201), as discussed in the explanation of FIG. 3(b), may include a plurality of links (301) connected together at joints (303). The robotic arm (201) has appropriately positioned electric motors (not shown) to provide for feedback. Furthermore, appropriately positioned positional sensors, e.g., encoders, or potentiometers, or the like, are positioned on the joints (303) so as to enable joint positions of a master control to be determined. Axes X, Y, and Z indicate the positional degrees of freedom of the robotic arms (201). In general, movement about joints of the links (301) primarily accommodates and senses orientational movement of an end effector attached to the tool interface assembly (200), and movement about the joints of robotic arm (201) primarily accommodates and senses translational movement of the end effector attached to the tool interface assembly (200).

In an embodiment, each robotic arm (201) may be operatively connected to one or more of the master controls so that the movement of the tool interface assembly (200) mounted on the robotic arm (201) is controlled by manipulation of the master controls. The tool interface assembly (200) carried on the robotic arm (201) have end effectors, which are mounted distal ends of elongated shafts of the tool interface assembly (201).

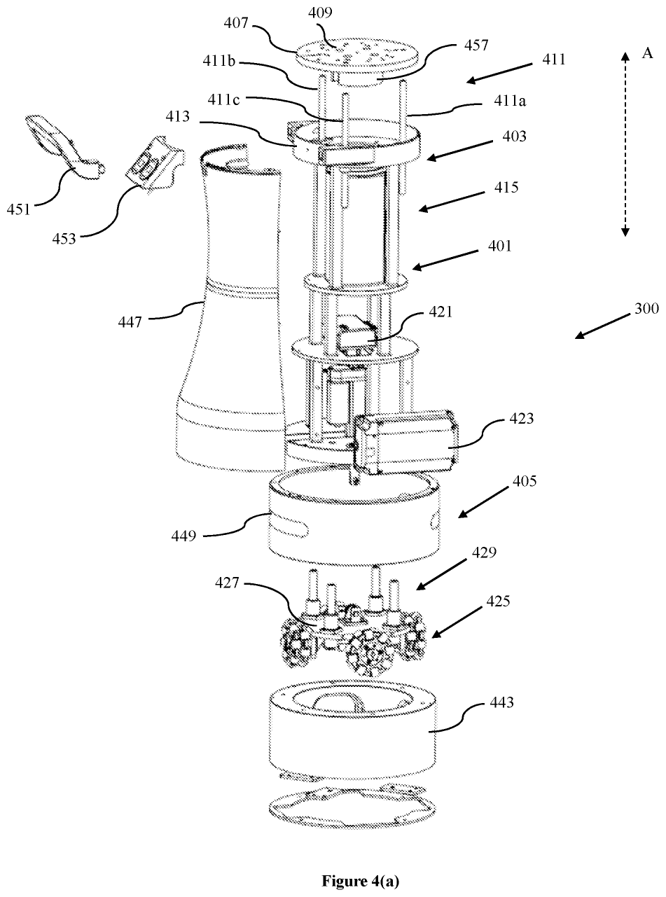

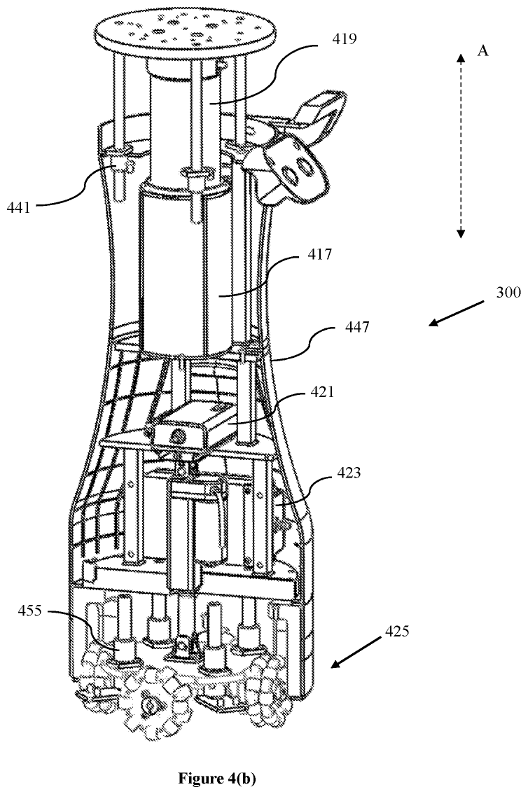

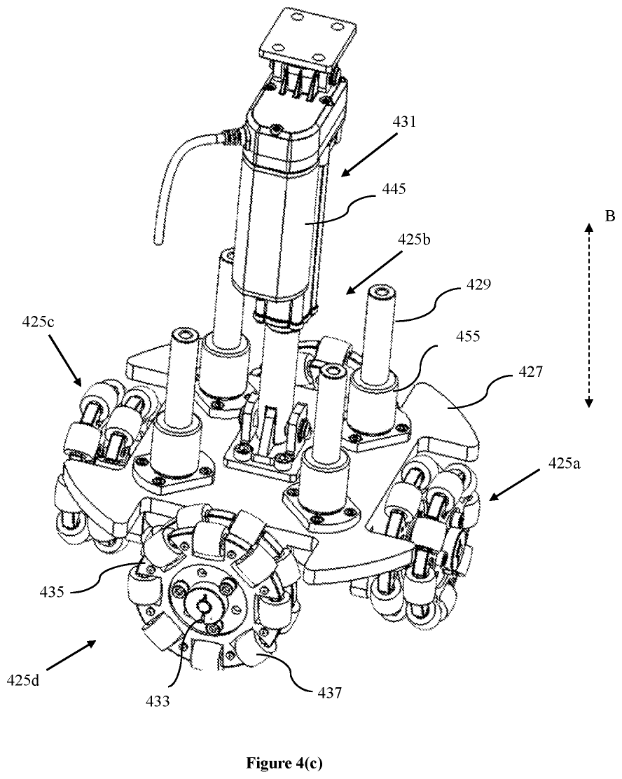

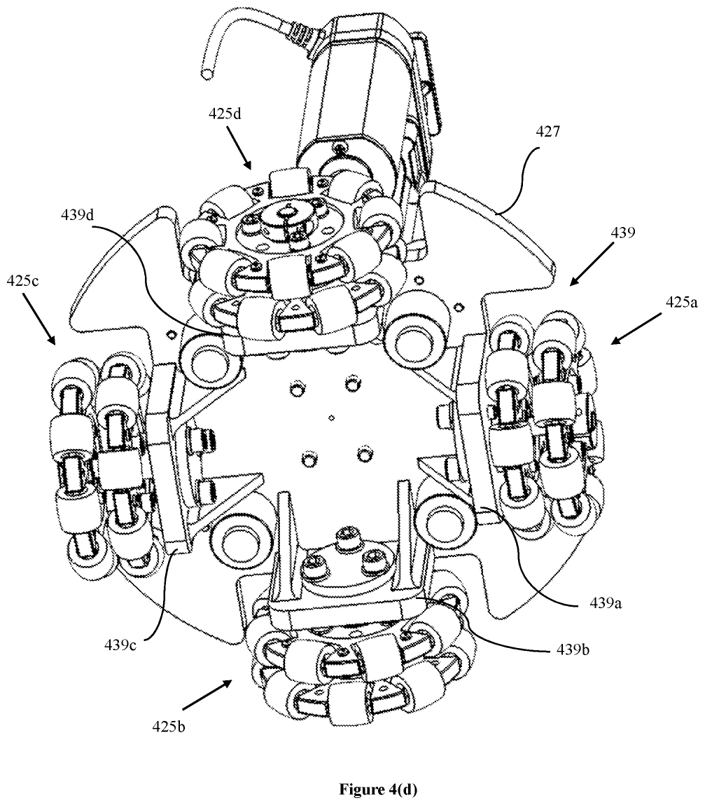

FIG. 4(a) illustrates an exploded view of the robotic surgical cart in accordance with an embodiment of the disclosure. The robotic surgical cart (300), as illustrated in FIG. 3(c), exemplifies a robotic arm (201) being configured to be capable of being releasably mounted on the robotic surgical cart (300) by means of various locking mechanisms, but not limited to bolts, snap fit, push button locking mechanism and the like.

One embodiment of the disclosure discloses the robotic surgical cart (300) may comprises a column (401) having a first end (403) and a second end (405). Detailed explanation of the column is provided in description of accompanying figures.

The robotic surgical cart (300) further may comprises a top plate (407) affixed on the first end (403) of the column (401). The top plate (407) may comprise of a cylindrical profile with guiding slots (409) on its inner circumference. The guiding slots (409) may be configured to received portions of the robotic arm (201) which may assist the mounting of the robotic arm (201) on the robotic surgical cart (300). The guiding slots (409) may be rectangular or circular in shape.

The top plate (407) may be made of any suitable resilient material such as a metal or an alloy. The material for the top plate (407) may be selected from a group consisting aluminum, steel, iron, nickel, copper, zinc, tin, or any combination thereof. In accordance to a specific embodiment of the disclosure, the top plate (407) is made of aluminum. The top plate (407) may be painted or may have a protective coating such as alloy coating. In accordance with an embodiment, the process of anodizing may be used to coat the top plate (407) such as to form a protective coating of aluminum oxide on the surface of the top plate (407). The top plate (407) may be of any suitable size that can be conveniently attached to the robotic arm (201) without affecting ease of the surgical operation. The top plate (407) may be of a suitable thickness providing sufficient strength.

The top plate (407) may be of any suitable shape such that the ease of affixing the robotic arm (201) is maintained. In accordance with an embodiment of the disclosure, the top plate (407) is substantially of a circular shaped plate where the top plate (407) is configured to consist of multiple openings on the outer periphery of the top plate (407). Also, the top plate (407) may be configured to comprises circular shaped protrusion (457) at its bottom end for attaching to another component of the robotic surgical cart (300), which is explained below.

Now referring to FIG. 4(b) along with FIG. 4(a), the robotic surgical cart (300) may further comprises at least one guiding shaft (411) affixed to the top plate (407) on one end to provides a controlled channel for movement of the robotic arm (201).

In an embodiment, there are three guiding shafts (411a), (411b), and (411c) attached to the top plate (407). These guiding shafts (411a), (411b), and (411c) are spaced apart from each other and are attached on one end to the base of the top plate (407). The guiding shafts (411a), (411b), and (411c) are connected to a circular band (413) at the other end with the help of bushings (441). Each of the three guiding shafts (411a), (411b), and (411c) are connected to the circular band (413) with the help of respective bushing (441).

In an extended state, shown in FIG. 4(b), the guiding shafts (411a), (411b), and (411c) are configured to provide upward/downwards movement such that the robotic arm (201) connected to the top plate (407) moves in a straight channel. Alternatively, in a docked position, shown in FIG. 3(c), the guiding shafts (411a), (411b), and (411c) are in contracted state such that the top plate (407) rests on the circular band (413).

The robotic surgical cart (300) may further comprises a telescopic pillar (415) including two or more tubular members, affixed below the top plate (407) in the column (401). The telescopic pillar (415) defines a longitudinal axis (A) to provide upward and downward movement of the two or more tubular members in the channel provided by the guiding shafts (411a), (411b), (411c) such that the telescopic pillar (415) moves the robotic arm (201) attached to the top plate (407) in a position in which a portion of the robotic arm (201) is exposed to contact a patient on a surgical table.

In an embodiment, the telescopic pillar (415) consists of two tubular members (417), (419) coaxial with one another and telescopically engaged one inside the other, that define a longitudinal axis of displacement (A) for the extension and withdrawal of the inner tubular member (419) in relation to the outer tubular member (417), thereby extending or withdrawing the robotic arm (201) attached to the top plate (407).

The embodiment described herein involves a telescopic pillar (415) comprising two tubular members (417), (419), but in other embodiments the telescopic pillar (415) may clearly comprise more than two coaxial tubular members.

The two tubular members (417), (419) may be associated with a drive unit (not shown) contained inside any of the tubular members (417), (419). The drive unit may also be associated with and operatively connected to means for controlling the displacement to enable the controlled extension or withdrawal of the inner tubular member (419).

In an embodiment, one of the two tubular members (417), (419) is configured to be attached on one end with the circular shaped protrusion (457) of the top plate (407) by means of various locking mechanisms, but not limited to bolts, snap fit, push button locking mechanism and the like.

In an embodiment, the telescopic pillar (415) may comprise of a safety device (not shown) operatively cooperating with the control means to stop the drive unit during the withdrawal of the tubular members (417), (419) in the event of any counteracting elements obstructing the lowering of the top plate (407).

The telescopic pillar (415) may be made of any suitable resilient material such as a metal or an alloy. The material for the telescopic pillar (415) can be selected from a group consisting aluminum, steel, iron, nickel, copper, zinc, tin, or any combination thereof. In accordance to a specific embodiment of the disclosure, the telescopic pillar (415) is made of aluminum. The telescopic pillar (415) may be painted or may have a protective coating such as alloy coating. In accordance with an embodiment, the process of anodizing may be used to coat the telescopic pillar (415) such as to form a protective coating of aluminum oxide on the surface of the telescopic pillar (415). The telescopic pillar (415) may be of any suitable size that can be conveniently attached to the column (401) of the robotic surgical cart (300) without affecting ease of the surgical operation. The telescopic pillar (415) may be of a suitable thickness providing sufficient strength.

The telescopic pillar (415) may be of any suitable shape such that the ease of affixing the telescopic pillar (415) in the column (401) is maintained. In accordance with an embodiment of the disclosure, the telescopic pillar (415) is substantially of a cylindrical shaped tube where the two tubular members (417), (419) are positioned one inside the other and are telescopically engaged one inside the other. According to an embodiment, both the tubular members (417), (419) are configured to extend and withdraw in respect of the other tubular member.

In an embodiment, the guiding shafts (411a), (411b), and (411c) and the telescopic pillar (415) work simultaneously to drive the robotic arm (201) upward and downward in the channel. In another embodiment, the two tubular members (417), (419) are configured to move upward and downward within a range of 1 mm to 150 mm.

The robotic surgical cart (300) further may comprises a control box (421) affixed within the column (401), wherein the control box (421) is configured to provide power to the telescopic pillar (415). In an embodiment, the control box (421) is configured to be affixed to the column (401) by means of various locking mechanisms, but not limited to bolts, snap fit, push button locking mechanism and the like.

In an embodiment, the control box (421) may be made of any suitable resilient material such as a metal or an alloy. The material for the control box (421) can be selected from a group consisting aluminum, steel, iron, nickel, copper, zinc, tin, or any combination thereof. In accordance to a specific embodiment of the disclosure, the control box (421) is made of aluminum. The control box (421) may be of any suitable shape such that the ease of affixing the control box (421) in the column (401) is maintained. In accordance with an embodiment of the disclosure, the control box (421) is substantially of a rectangular shaped box where the control box (421) is connected to the telescopic pillar (415) by means of various connection mechanism, but not limited to a wire.

The robotic surgical cart (300) may further comprise of a secondary battery (423) affixed to the second end (405) of the column (401), wherein the secondary battery (423) acts as a backup battery to power the telescopic pillar (415) in case of a failure of the control box (421). In an embodiment, the secondary battery (423) is configured to be affixed to the second end (405) of the column (401) by means of various locking mechanisms, but not limited to bolts, snap fit, push button locking mechanism and the like.

In an embodiment, a box (not shown) enclosing the secondary battery (423) is provided and may be made of any suitable resilient material such as a metal or an alloy. The material for the box containing the secondary battery (423) can be selected from a group consisting aluminum, steel, iron, nickel, copper, zinc, tin, or any combination thereof. In accordance to a specific embodiment of the disclosure, the box enclosing secondary battery (423) is made of steel. The box enclosing secondary battery (423) may be of any suitable shape such that the ease of affixing the secondary battery (423) in the column (401) is maintained. In accordance with an embodiment of the disclosure, the secondary battery (423) is substantially of a rectangular shaped battery which is connected to the telescopic pillar (415) by means of various connection mechanism, but not limited to a wire or any electrical connection.

Referring now to FIG. 4(c), the robotic surgical cart (300) may further comprises a set of omni-directional wheels (425) affixed to a base plate (427) at the second end (405) of the column (401). In an embodiment, the robotic surgical cart (300) may comprises four omni-directional wheels (425a), (425b), (425c), and (425d) with each of the omni-directional wheels (425a), (425b), (425c), and (425d) having another supportive wheel attached together to form one set.

In an embodiment, each of the omni-directional wheel (425a), (425b), (425c), and (425d) comprises a hub (433), a plurality of roller mounting brackets (435) coupled to the hub (433), and a plurality of rollers (437) each rotatably coupled to at least one of the roller mounting brackets (435).

The hub (433) is configured to support the plurality of rollers (437) on multiple the roller mounting brackets (435). The hub (433) is positioned centrally within the omni-directional wheel (425a), (425b), (425c), and (425d) and is mounted to a wheel axle (not shown) which is coupled to the base plate (427). The wheel axle may be a cylindrical tube attached to a portion of base plate (427) at one end and to the hub (433) on the other end by means of various locking mechanisms, but not limited to bolts, snap fit, push button locking mechanism and the like.

The plurality of rollers (437) are coupled to the hub (433) by roller mounting brackets (435) in fixed positions about an outer periphery of the hub (433) such that an axle (not shown) of the roller are at a fixed angle with respect to the wheel axle i.e., substantially perpendicular angle. Alternatively, an acute angle may be formed by projecting a centerline of the axle of the roller onto the center line of the wheel axle is defined as a roller mounting angle. Each of the omni-directional wheels (425a), (425b), (425c), and (425d) may be designed with roller mounting angles of between approximately 20 degrees and 90 degrees, but roller mounting angles of approximately 45 degrees and 90 degrees are most commonly used in practice.

In an embodiment, each of the omni-directional wheels (425a), (425b), (425c), and (425d) can have another support wheel attached to them such that two rows of wheels are positioned on each wheel axle attached to the base plate (427), as seen in FIG. 4(c). The configuration of both the wheels are the same.

In another embodiment, the number of rollers (437) on each of the omni-directional wheel (425a), (425b), (425c), and (425d) is variable from a minimum of four to eight rollers on a wheel. In an embodiment, each of the omni-directional wheel (425a), (425b), (425c), and (425d) has eight rollers on each wheel making it sixteen rollers in one set. In another embodiment, the circular wheel profile as shown in FIG. 4(c), depicts the omni-directional wheels (425a), (425b), (425c), and (425d) with sixteen rollers group at a 90-degree angle to the wheel axle.

The rollers (437) have a flexible ground contacting material typically made from an elastomer such as rubber or urethane. The roller (437) ground contacting surface may have a convexly vaulted exterior profile or a circular profile that may be based upon the number of rollers (437) mounted on the hub (433), a diameter of the omni-directional wheel (425), a roller (437) center diameter, and the roller angle such that when the omni-directional wheel (425) turns its contact with the ground shifts from roller to roller in a continuous fashion.

The roller (437) contacting surface is made of a flexible material that will deflect at the point of contact with the ground to spread the applied load onto a finite area on the ground. The roller (437) contacting surface may be made of an elastomer, such as urethane or natural rubber, which will have the added benefit of providing traction with the ground surface. The elastomer may be reinforced with fibers such as fiberglass and friction-enhanced with materials such as carbon black. Additionally, other materials may be used for higher load applications, such as glass filled nylon.

In an embodiment, when an omni-directional wheels (425) supports the weight of a medical cart (300), the weight is transmitted through the wheel axle to the hub (433), then through the roller mounting bracket (435) to the roller (437) which transmits the weight to core of the one or more roller (437) and through it to one or more rollers (437) whose surface is in contact with the ground, where the weight is applied to the ground.

In use, the omni-directional robotic surgical cart (300), shown in FIG. 3(a) or 3(b), is capable of moving in any direction due to the interplay between the rollers (437) and the omni-directional wheels (425). Usually, the omni-directional wheels (425) are unique as they are able to roll freely in two directions as ether roll like a normal wheel or roll laterally using the wheels along its circumference.

FIG. 4(d) illustrates the bottom view of the omni-directional wheels attached to the base plate in accordance with an embodiment of the disclosure. The base plate (427) is shaped in a fashion where the base plate (427) being a flat circular plate having portions (439) inwardly positioned at least 90 degrees from the horizontal base plate (427) such that the omni-directional wheels (425a), (425b), (425c), and (425d) can be affixed to the portions (439) by means of various locking mechanisms, but not limited to bolts, snap fit, push button locking mechanism and the like.

In an embodiment, the omni-directional wheels (425a), (425b), (425c), and (425d) is affixed to the respective portions (439a), (439b), (439c), and (439d) of the base plate (427). In an embodiment, the base plate (427) may be made of any suitable resilient material such as a metal or an alloy. The material for the base plate (427) can be selected from a group consisting aluminum, steel, iron, nickel, copper, zinc, tin, or any combination thereof. In accordance to a specific embodiment of the disclosure, the base plate (427) is made of steel.

Referring back to FIG. 4(c), the robotic surgical cart (300) may further comprises at least one guiding shaft (429) affixed to the base plate (427) at one end and to the column (401) at the other end. The at least one guiding shaft (429) is configured to provide a controlled channel for movement of the group of omni-directional wheels (425). According to an embodiment, there are four guiding shafts (429a), (429b), (429c), and (429d) attached to the base plate (427) and thereon to the chamber (401).

The guiding shafts (429a), (429b), (429c), and (429d) are spaced apart from each other and are attached on one end to the base plate (427) with help of bushings (455) and further bolted thereon to the base plate (427). The guiding shafts (429a), (429b), (429c), and (429d) are connected on the other end to the chamber (401) with the help of bolts (not shown).

In an extended state, as shown in FIG. 4(b), the guiding shafts (429a), (429b), (429c), and (429d) are configured to move the omni-directional wheels (425) connected to the base plate (427) in a straight channel and protrude visibly outwards of the footprint ring (443). Alternatively, in a docked position, the guiding shafts (429a), (429b), (429c), and (429d) are in contracted state such that omni-directional wheels (425) are not visible and rests inwards with the footprint ring (443).

Referring now to FIG. 4(c), the robotic surgical cart (300) may further comprises an actuator (431) connected to the base plate (427) at one end and connected to the chamber (401) at the other end. The actuator (431) defining an axis (B) to provide upward and downward movement of the set of omni-directional wheels (425) in the channel provided by the at least one guiding shaft (429). In an embodiment, the actuator (431) is a linear actuator.

The actuator (431) connected to the base plate (427) at one end and connected to the chamber (401) at the other end. The actuator (431) defining the axis (B) to provide upward and downward movement of the set of omni-directional wheels (425) in the channel provided by the at least one guiding shaft (429) such that the actuator (431) moves the set of omni-directional wheels (425) attached to the base plate (427) in a position in which the set of omni-directional wheels (425) protrude outwards from a contracted state. The actuator (431) moves the set of omni-directional wheels (425) in between two thresholds or two end-points thereby providing adjustment to height of the robotic surgical cart (300).

In an embodiment, the actuator (431) is configured to be attached on one end with the base plate (427) by means of various locking mechanisms, but not limited to bolts, snap fit, push button locking mechanism and the like. Also, the actuator (431) is configured to be attached on the other end with one section of the chamber (401) by means of various locking mechanisms, but not limited to bolts, snap fit, push button locking mechanism and the like.

In an embodiment, the actuator (431) may comprises a housing (445) which may include an electric motor (not shown) that is mechanically connected to rotate a lead screw (not shown). The lead screw may have a continuous helical thread machined on its circumference running along its length. A lead nut or a ball nut may be threaded onto the lead screw with corresponding helical threads. The lead nut is prevented from rotating with the lead screw and the lead nut interlocks with a non-rotating part of the actuator housing. Therefore, when the lead screw is rotated, the lead nut will be driven along the threads. The direction of motion of the lead nut depends on the direction of rotation of the lead screw. By connecting linkages to the lead nut, the motion can be converted to usable linear displacement and thereby moving the set of omni-directional wheels (425) in an upward or downward linear direction.

In another embodiment, many types of motors can be used in an actuator system. These include DC Brush, DC Brushless, Stepper, or Induction Motors. The housing (445) of the actuator (431) may be made of any suitable resilient material such as a metal or an alloy. The material for the housing (445) can be selected from a group consisting aluminum, steel, iron, nickel, copper, zinc, tin, or any combination thereof. In accordance to a specific embodiment of the disclosure, the housing (445) is made of aluminum or steel.

The actuator (431) may be of any suitable shape such that the ease of affixing the actuator (431) in the column (401) is maintained. In accordance with an embodiment of the disclosure, the actuator (431) is substantially of a cylindrical shaped tube where the motors and the lead screw and lead nut are positioned.

In an embodiment, the guiding shafts (429a), (429b), (429c), and (429d) and the actuator (431) work simultaneously to drive the set of omni-directional wheels (425) upward and downward in the channel. In another embodiment, the set of omni-directional wheels (425) are configured to move upward and downward within a range of 1 mm to 50 mm.

Referring back to FIG. 4(a), the robotic surgical cart (300) may further comprise of an outer casing (447) enclosing the column (401) and the other components of the robotic surgical cart (300). The outer casing (447) is configured to cover the all the components of the robotic surgical cart (300) to provide an ascetic look. The outer casing (447) is configured to be attached to the column (401) by means of various locking mechanisms, but not limited to bolts, snap fit, push button locking mechanism and the like.

In an embodiment, the outer casing (447) may be made of any suitable resilient material such as a metal or an alloy. The outer casing (447) can be selected from a group consisting aluminum, steel, iron, nickel, copper, zinc, tin, or any combination thereof. In accordance to a specific embodiment of the disclosure, the outer casing (447) is made of steel. The outer casing (447) may be of any suitable shape such that the ease of affixing the outer casing (447) in the column (401) is maintained. In accordance with an embodiment of the disclosure, the outer casing (447) is substantially of a concave shape where the top portion of the outer casing (447) is substantially tapered than the bottom portion of the outer casing (447).

The robotic surgical cart (300) may further comprise of a footprint ring (443) affixed on the second end (405) of the column (401), wherein the footprint ring (443) provides a stable platform to the robotic surgical cart (300). The footprint ring (443) is circular in shape and encloses the omni-directional wheels (425) at the bottom of the robotic surgical cart (300). In an embodiment, the footprint ring (443) may be made of any suitable resilient material such as a metal or an alloy. The footprint ring (443) can be selected from a group consisting aluminum, steel, iron, nickel, copper, zinc, tin, or any combination thereof. In accordance to a specific embodiment of the disclosure, the footprint ring (443) is made of steel. In another embodiment, another set of outer casing (449) is configured to enclose the footprint ring (443).

The robotic surgical cart (300) may further comprise of a handle bar (451) pivotally affixed to the first end (403) of the column (401), where the outer casing (447) may enclose the column (401). The handle bar (451) has a U-shaped structure and configured to allow a user to move the robotic surgical cart (300) from one position to another position. This permits the hand of the user of the robotic surgical cart (300) gripping a grip portion to automatically move to its most natural or strain free position as the user pulls or pushes the robotic surgical cart (300). The handle bar (451) includes two molded or cast halves (not shown), preferably of a strong, tough plastic material such as, for example, nylon. Bolts are used to secure the handle halves together and onto the outer casing (447).

The robotic surgical cart (300) may further comprise of a user interface (453) pivotally affixed to the outer casing (447), where the outer casing (447) encloses the first end (403) of the column (401), wherein the user interface (453) provides the controls for the movement of the robotic arm (201) and wherein the user interface (453) comprises a screen displaying the height of the robotic surgical cart and the distance from the surgical table.

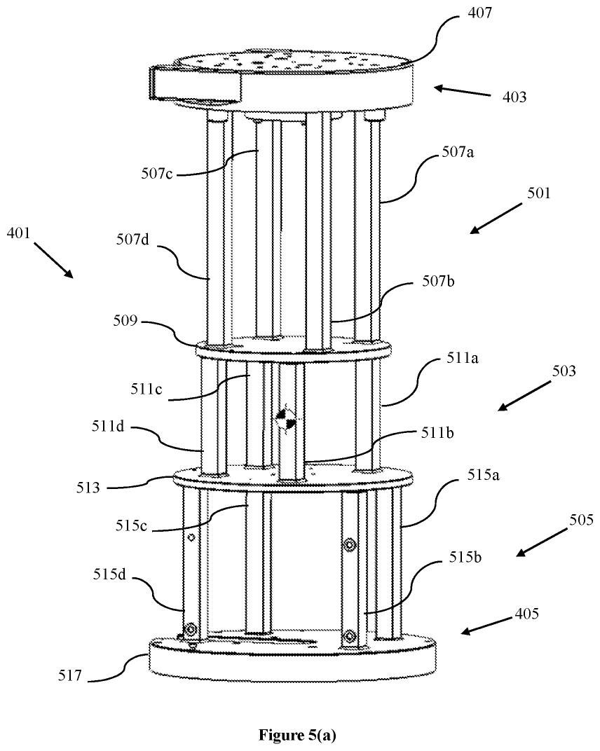

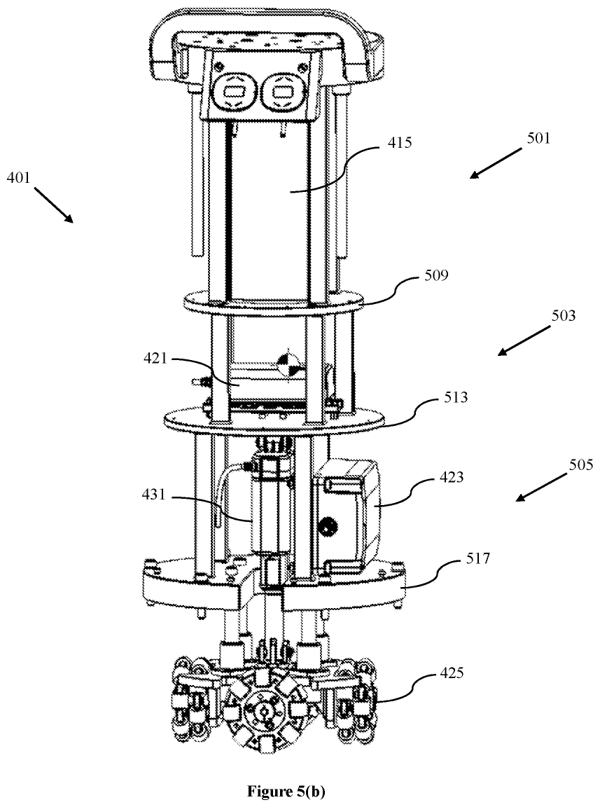

FIG. 5(a) illustrates a perspective view of a column in accordance with an embodiment of the disclosure. The column (401) comprises at least one shaft and at least one stabilizing plate joined together with the shaft at an end.

In an embodiment, the column (401) comprises three compartments (501), (503), and (505), where the first compartment (501) comprises four shafts (507a), (507b), (507c), and (507d) connected to the top plate (407) at one end and to a first stabilizing plate (509) at other end, by means of various locking mechanisms, but not limited to bolts, welding, snap fit, push button locking mechanism and the like. In an embodiment, the four shafts (507a), (507b), (507c), and (507d) are welded to the top plate (407) and the first stabilizing plate (509).

In another embodiment, the second compartment (503) comprises four shafts (511a), (511b), (511c), and (511d) connected to the first stabilizing plate (509) at one end and to a second stabilizing plate (513) at other end, by means of various locking mechanisms, but not limited to bolts, welding, snap fit, push button locking mechanism and the like. In an embodiment, the four shafts (511a), (511b), (511c), and (511d) are welded to the first stabilizing plate (509) and the second stabilizing plate (513).

In another embodiment, the third compartment (505) comprises four shafts (515a), (515b), (515c), and (515d) connected to the second stabilizing plate (513) at one end and to a third stabilizing plate (517) at other end, by means of various locking mechanisms, but not limited to bolts, welding, snap fit, push button locking mechanism and the like. In accordance with a specific embodiment, the four shafts (515a), (515b), (515c), and (515d) are welded to the second stabilizing plate (513) and the third stabilizing plate (517). The third stabilizing plate (517) comprises a recess (519) to receive the housing (445) of the actuator (431).

The column (401) may be made of any suitable resilient material such as a metal or an alloy. The material for the column (401) may be selected from a group consisting aluminum, steel, iron, nickel, copper, zinc, tin, or any combination thereof. In accordance to a specific embodiment of the disclosure, the column (401) is made of steel. The column (401) may be painted or may have a protective coating such as alloy coating. In accordance with an embodiment, the process of anodizing may be used to coat the column (401) such as to form a protective coating of aluminum oxide on the surface of the column (401). The column (401) may be of any suitable size that can be conveniently attached within the robotic surgical cart (300) without affecting ease of the surgical operation. The column (401) may be of a suitable thickness providing sufficient strength.

The column (401) may be of any suitable shape such that the ease of affixing the robotic arm (201) is maintained. In accordance with an embodiment of the disclosure, the stabilizing plates (509), (513), and (517) are substantially of a circular shaped plate where the aforesaid stabilizing plates have configuration to attach multiple components of the robotic surgical cart (300). The aforesaid shafts are substantially of a pipe like structure and is configured to be attached to the respective stabilizing plates.

FIG. 5(b) illustrates various components positioned in various compartments in accordance with an embodiment. The first compartment (501) contains the telescopic pillar (415) which is attached to the top plate (407) on one end and to the first stabilizing plate (509) at other end by an attachment means such as bolts.

In another embodiment, the second compartment (503) contains the control box (421) which is attached to the second stabilizing plate (513) at other end by an attachment means such as bolts.

In another embodiment, the third compartment (505) contains the actuator (431) which is attached to the second stabilizing plate (513) on one end and to the base plate (427) at other end by an attachment means such as bolts. Also, the third compartment (505) holds the secondary battery (423) attached to the third stabilizing plate (517) at one end by an attachment means such as bolts.

The foregoing descriptions of exemplary embodiments of the present disclosure have been presented for purposes of illustration and description. They are not intended to be exhaustive or to limit the disclosure to the precise forms disclosed, and obviously many modifications and variations are possible in light of the above teaching. The exemplary embodiment was chosen and described in order to best explain the principles of the disclosure and its practical application, to thereby enable others skilled in the art to best utilize the disclosure and various embodiments with various modifications as are suited to the particular use contemplated. It is understood that various omissions, substitutions of equivalents are contemplated as circumstance may suggest or render expedient, but is intended to cover the application or implementation without departing from the spirit or scope of the claims of the present disclosure.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

While specific language has been used to describe the disclosure, any limitations arising on account of the same are not intended. As would be apparent to a person in the art, various working modifications may be made to the apparatus in order to implement the inventive concept as taught herein.

We claim:

1. A robotic surgical cart (300) for a robotic arm (201), comprising:
a column (401) having a first end (403) and a second end (405),
a top plate (407) affixed on the first end (403) of the column (401), wherein the top plate (407) is configured to mount the robotic arm (201) thereon;
at least one guiding shaft (411) affixed on one end to the top plate (407), that provides a controlled channel for movement of the robotic arm (201); and
a telescopic pillar (415) including two or more tubular members (417, 419), affixed below the top plate (407); the telescopic pillar (415) defining a longitudinal axis to provide upward and downward movement of the two or more tubular members (417, 419) in the channel provided by the at least one guiding shaft (411) such that the telescopic pillar (415) moves the robotic arm (201) in a position in which a portion of the robotic arm (201) is exposed to contact a patient on a surgical table.

2. The robotic surgical cart (300) as claimed in claim 1, wherein the two or more tubular members (417, 419) coaxial with one another and telescopically engaged one inside the other.

3. The robotic surgical cart (300) as claimed in claim 1, wherein the two or more tubular members (417, 419) are configured to move upward and downward within a range of 1 mm to 150 mm.

4. The robotic surgical cart (300) as claimed in claim 1, further comprising a user interface (453) pivotally affixed to the first end (403) of the column (401), wherein the user interface (453) provides the controls for the movement of the robotic arm (201) and wherein the user interface (453) comprises a screen displaying the height of the robotic surgical cart (300) and the distance from the surgical table.

5. The robotic surgical cart (300) as claimed in claim 1, wherein the column (401) comprises at least one shaft and at least one stabilizing plate joined together with the shaft at an end.

6. The robotic surgical cart (300) as claimed in claim 1, further comprising a control box (421) affixed within the column (401), wherein the control box (421) is configured to provide power to the telescopic pillar (415).

7. The robotic surgical cart (300) as claimed in claim 1, further comprising a secondary battery (423) affixed to the second end (405) of the column (401), wherein the secondary battery (423) is a backup battery to power the telescopic pillar (415) in case of a failure of the control box (421).

8. The robotic surgical cart (300) as claimed in claim 1, further comprising an outer casing (447) enclosing the column (401) and the other components (415, 421, 423) of the robotic surgical cart (300).

9. The robotic surgical cart as claimed in claim 1, further comprising a handle bar (451) pivotally affixed to the first end (403) of the column (401), wherein the handle has a U-shaped structure and configured to allow a user to move the robotic surgical cart (300) from one position to another position.

10. The robotic surgical cart (300) as claimed in claim 1, further comprising a footprint ring (443) affixed on the second end (405) of the column (401), wherein the footprint ring (443) provides a platform to the robotic surgical cart (300).
